(19) United States
(12) Patent Application Publication
(10) Pub. No.: US 2006/0292225 A1
(43) Pub. Date: Dec. 28, 2006

(54) WATER SOLUBLE ANALGESIC FORMULATIONS AND METHODS FOR PRODUCTION

(76) Inventors: Arthur M. FELIX, West Caldwell, NJ (US); Robert E. Martin, Long Beach, NY (US)

Correspondence Address: NOTARO AND MICHALOS, 100 DUTCH HILL ROAD, SUITE 110, ORANGEBURG, NY, 10962-2100, US

(21) Appl. No.: 11/424,560

(22) Filed: Jun. 16, 2006

Related U.S. Application Data
(60) Provisional application No. 60/693,591, filed on Jun. 24, 2005.

(51) Int. Cl. A61K 31/60; A61K 9/14

(52) U.S. Cl. 424/489; 514/165

(57) ABSTRACT

A water soluble analgesic composition includes a plurality of granules. Each of the granules includes a substrate core and a coating disposed on the substrate core forming an agglomerated product, the coating including a salt of an analgesic, but substantially no particles of a non-salt form of the analgesic. The composition may be created by a method including the steps of: (i) providing a first solution comprising a base, (ii) adding an analgesic to the first solution to create a second solution including a salt of the analgesic, (iii) filtering the second solution to remove residual particles of the analgesic to create a filtered second solution, and (iv) spray drying the filtered second solution onto a substrate to form an agglomerated product having a plurality of granules.

### RELATED APPLICATIONS

This patent application claims the benefit of, under Title 35, United States Code, Section 119(e), U.S. Provisional Patent Application No. 60/693,591, filed Jun. 24, 2005.

### FIELD OF THE INVENTION

The present invention relates generally to aspirin and other analgesic compositions and, more specifically, to water soluble aspirin and other analgesic compositions which have enhanced stability and bioactivity as compared to previously known water soluble aspirin and other analgesic compositions.

### BACKGROUND OF THE INVENTION

Acetylsalicylic acid (aspirin), an important member of a family of therapeutics known as non-steroidal anti-inflammatory drugs (NSAIDs) is known to have analgesic, antipyretic and anti-inflammatory properties. These multiple properties make it an ideal therapeutic for pain relief (including, but not limited to, the treatment of headaches), fever reduction and treatment of arthritis and other related indications. Aspirin's mechanism of action involves the inhibition of the synthesis of prostaglandins from arachidonic acid. Aspirin acetylates a serine residue in the active site of PGH2 synthase, the enzyme that catalyzes the conversion of arachidonic acid to PGH2. This acetylation of PGH2 synthase inhibits the action of the enzyme and, therefore, inhibits prostaglandin synthesis.

In the last 50 years, aspirin has also been shown to have remarkable antithrombotic benefits. Aspirin's antithrombotic effect is mediated by inhibition of blood platelets. The drug blocks a platelet enzyme, cyclo-oxygenase, by acetylating the enzyme's active site. Inhibition of the enzyme blocks production of an important prothrombotic agent known as thromboxane A2. Thromboxane A2 causes activation and aggregation of platelets, which is an early step in thrombosis. Today, several platelet inhibitors are available, but aspirin remains the most commonly used drug in this category and is still a very cost-effective antithrombotic drug. Aspirin (either 81 mg or 325 mg daily) is indicated in the following conditions: unstable angina (acute coronary syndrome), acute myocardial infarction, secondary prevention of myocardial infarction, secondary prevention of stroke (carotid or primary cerebrovascular disease), prevention of peripheral arterial thrombosis, and prevention of venous thrombosis (deep venous thrombosis, pulmonary embolism). There has also been investigation recently of using aspirin (either alone or in combination with other medications) for the treatment of various types of cancer.

The pharmacokinetic properties (absorption, distribution, metabolism and elimination) of aspirin are important. Absorption of aspirin following enteral administration involves passage through appropriate membranes into the plasma.

The degree of absorption is related to solubility, dosage form, excipients and particle size. In general, lipid-soluble, undissociated forms of a drug readily pass through membranes. Ionization of aspirin is suppressed in the stomach (low pH); therefore aspirin is absorbed into the bloodstream in significant quantities in its unionized (uncharged) form through the stomach membrane. The main metabolic pathway for aspirin is via esterase-catalyzed hydrolysis to salicylic acid which is unable to inhibit the synthesis of prostaglandins.

Although aspirin has been reported to be useful in a variety of pathophysiological settings, ranging from low doses for heart-attack and stroke prevention to high doses for rheumatoid arthritis, its application has been limited due to its poor solubility in water. Side-effects stemming from undissolved particles that can adhere to gastrointestinal mucosa may cause gastric or intestinal ulceration and bleeding that may lead to anemia from resultant blood loss.

More specifically, the common dosages of aspirin (325 mg or 500 mg), are generally considered adequate for “aspirin therapy” to reduce the likelihood of heart-attack and/or stroke. However, these dosages only provide relief of the symptom of arthritis (i.e., pain), and do not treat the underlying inflammation. In order to achieve effective control of inflammation, the cause of arthritis, daily dosages of 4,000 to 5,000 mg or greater are generally needed to maintain plasma salicylate concentrations in the range of 120 to 350 μg/ml. At these higher dose levels, the rate of successful treatment is over 70%. However, the success rate falls off dramatically at lower daily dosages, and with 2500 mg, for example, it is less than 10%. Thus, the cause of failure, or the lack of success, with aspirin therapy in the context of treating arthritis inflammation may be due, at least in part, to the use of inadequate dosages.

Unfortunately, aspirin exhibits a number of undesirable side effects. The most commonly experienced side effects are nausea, gastric upset (heartburn) and pain. At low analgesic dose levels these side effects will generally occur in about 2-10% of adult users of aspirin. However, this number increases dramatically with extended aspirin consumption. With higher anti-inflammatory dosages, the incidence of these undesirable side effects generally rises to about 25%. Again, this number increases significantly with extended treatment regimes.

The gastrointestinal side effects of aspirin are typically localized, and when aspirin is used in its current conventional form, as a suspension its undissolved particles tend to adhere to the stomach mucosa, causing irritation, inflammation and injury. The localized nature of these detrimental side effects has been established by gastroscopy and autopsies. Erosion, for example, around undissolved particles of aspirin in the stomach has been well documented and photographed. Because aspirin is a direct irritant to the gastrointestinal mucosa, its effects are both cumulative and persistent.

Localized side effects do not occur, however, when aspirin is administered in solution form. While all users of aspirin could benefit greatly from the advantages of its soluble form, older patients are in particular need of such a soluble aspirin product, because arthritis is a dreaded disease of old age. The elderly, as a group, are the largest users of aspirin and, at the same time, the most vulnerable to its acute side effects.

Due to reduced stomach motility and increased emptying time, which occur with aging, insoluble aspirin particles remain in contact with the stomach mucosa much longer in the elderly, thereby intensifying the undesirable side effects. In addition, it is estimated that there are more than 15 million people in the United States who experience some degree of difficulty in swallowing tablets and other solid medications. Older people, once again, are affected, as esophagus muscles weaken with age and make swallowing much more difficult.

Aspirin's low solubility in water and potential for hydrolysis have prevented its administration in aqueous solution, and therefore, aspirin is usually dispensed as tablets or capsules requiring large volumes of water to minimize gastric irritation. Aspirin is readily soluble in alkaline solution, but undergoes rapid hydrolysis to salicylic acid and acetic acid. In general, aspirin is more stable at lower pH, with maximum stability at pH 2.4.

As is generally well known, there have traditionally been some soluble aspirin products that are available commercially in the U.S. and in Europe. Unfortunately, they all suffer from one or more shortcomings that have prevented their universal acceptance, especially in the United States. For example, the only soluble product that is widely commercially available in the United States, Alka Seltzer®, distributed by Bayer HealthCare LLC, contains 567 mg of sodium per 325 mg of aspirin (1,750 mg of sodium per 1,000 mg of aspirin). In order to provide anti-inflammatory activity with Alka Seltzer®, daily ingestion of more than 8,000 mg of sodium would be required. This amount of sodium makes it totally unacceptable for regular aspirin therapy. Not only is this sodium level extremely high for the population in general, but it can not be tolerated by many of the elderly arthritic who are also on a restricted sodium diet. Even the levels of sodium associated with the lower dosages of aspirin that are effective to reduce the likelihood of heart-attack and/or stroke are unacceptably high.

In Europe, where drinkable analgesics dominate, most are fine suspensions, not true solutions. The majority of such products, like Alka Seltzer®, are sodium-based, take a relatively long time to dissolve and are not fully palatable. Some are calcium-based, thereby preventing total dissolution of the aspirin. A French soluble analgesic product, “Aspegic,” is also known. However, this product contains the unnatural dl-form of lysine, which might have difficulty winning FDA approval in the United States.

Numerous attempts have been made to produce an acceptable soluble aspirin product in the past, but none have proven to be totally satisfactory.

U.S. Pat. Nos. 5,665,388 and 5,723,453 to Phykitt, for example, disclose an essentially sodium free soluble alkaline aspirin compound. The formulations disclosed in these references, however, suffer from a number of disadvantages. One of such disadvantages is that the use of bicarbonates, as disclosed therein, causes gas to be formed when ingested by patients. Another disadvantage is that the relatively high pH of the compositions disclosed therein (i.e., greater than 8.0) leads to rapid hydrolysis and instability and, therefore, a shortened shelf-life.

U.S. Pat. Nos. 5,157,030 and 5,776,431 to Galat also disclose aspirin compounds, which aspirin compounds have disadvantages similar to those disclosed in the above-referenced prior art patents. Specifically, the compositions disclosed in these references have resulting pH values, when mixed with water, of over pH 6.0. This causes the compositions to be relatively unstable, have a shortened shelf-life, and be less readily absorbed by the body, since the aspirin component is in a less undissociated form. This also causes a relatively slow dissolution of the compositions in water, it having been found that compositions formulated in accordance with the Galat patents take up to two to three minutes to substantially completely dissolve in water. In addition, many of the formulations disclosed in the Galat patents are formed as two separate compositions (mixture “A” and mixture “B”), which is disadvantageous from manufacturing, packaging and use standpoints. Furthermore, the formulations in these references are blended and then directly added to water. There is no indication that the blended product is stable and can be packaged.

Therefore, at the present time, there is no satisfactory aspirin product available that is sodium free, that is rapidly water soluble, that is fast acting and enters the bloodstream rapidly, and that may be used in the relatively large dosages that are required for anti-inflammatory treatment, and/or that may be used for extended periods of time, without causing gastrointestinal upset and/or damage.

What is desired, therefore, is a water soluble analgesic composition which has enhanced stability and bioactivity as compared to previously known water soluble analgesic compositions, and which does not suffer from the disadvantages described above.

### SUMMARY OF THE INVENTION

Accordingly, it is an object of the present invention to provide a water soluble analgesic composition which has enhanced stability and bioactivity, as compared to previously known water soluble analgesic compositions.

Another object of the present invention is to provide a water soluble analgesic composition having the above characteristics and which is sodium free.

A further object of the present invention is to provide a water soluble analgesic composition having the above characteristics and which is rapidly water soluble.

Still another object of the present invention is to provide a water soluble analgesic composition having the above characteristics and which is fast acting and enters the bloodstream rapidly.

Yet a further object of the present invention is to provide a water soluble analgesic composition having the above characteristics and which may be used in the relatively large dosages that are required for antiinflammatory treatment, and/or that may be used for extended periods of time, without causing gastrointestinal upset and/or damage.

These and other objects of the present invention are achieved in accordance with one embodiment of the present invention by provision of a water soluble analgesic composition including a plurality of granules. Each of the granules includes a substrate core and a coating disposed on the substrate core forming an agglomerated product, the coating including a salt of an analgesic, but substantially no particles of a non-salt form of the analgesic.

In some embodiments, the substrate core is selected from the group consisting of monosaccharides, disaccharides, polysaccharides, dipeptides and combinations of these. In certain of these embodiments, the substrate core comprises sucrose. In some embodiments, the granules have a median diameter falling within a range from about 100μ to about 400μ. In certain of these embodiments, the granules have a median diameter of about 200μ. In some embodiments, the analgesic is selected from the group consisting of aspirin, 5-aminosalicylic acid, ibuprofen, naproxen, acetaminophen and combinations of these. In certain of these embodiments, the analgesic comprises aspirin. In some embodiments, the salt of the analgesic comprises a potassium salt of the analgesic.

In accordance with another embodiment of the present invention, a method of creating a water soluble analgesic composition includes the steps of: (i) providing a first solution comprising a base, (ii) adding an analgesic to the first solution to create a second solution including a salt of the analgesic, (iii) filtering the second solution to remove residual particles of the analgesic to create a filtered second solution, and (iv) spray drying the filtered second solution onto a substrate so as to form an agglomerated product comprising a plurality of granules.

In some embodiments, the analgesic is selected from the group consisting of aspirin, 5-aminosalicylic acid, ibuprofen, naproxen, acetaminophen and combinations of these. In certain of these embodiments, the analgesic comprises aspirin. In some embodiments, the base comprises tripotassium citrate monohydrate. In some embodiments, the first solution further comprises a surfactant. In certain of these embodiments, the surfactant comprises sodium lauryl sulfate. In some embodiments, the substrate is selected from the group consisting of monosaccharides, disaccharides, polysaccharides, dipeptides and combinations of these. In certain of these embodiments, the substrate comprises sucrose. In some embodiments, the step of spray drying the filtered second solution onto a substrate employs a fluid-bed spray drying process. In some embodiments, the granules have a median diameter falling within a range from about 100μ to about 400μ. In certain of these embodiments, the granules have a median size of about 200μ.

In accordance with a further embodiment of the present invention, a water soluble analgesic composition includes aspirin and tripotassium citrate monohydrate, with the aspirin comprising at least about 26% by weight of a combined weight of the aspirin and the tripotassium citrate monohydrate.

In some embodiments, the aspirin comprises from about 26% to about 40% by weight of a combined weight of the aspirin and the tripotassium citrate monohydrate. In some embodiments, a pH of the composition, when dissolved in water, is below about 6.0.

In some embodiments, the water soluble analgesic composition further includes a substrate. In certain of these embodiments, the substrate is selected from the group consisting of monosaccharides, disaccharides, polysaccharides, dipeptides and combinations of these. In certain of these embodiments, the substrate comprises sucrose. In some embodiments, the substrate comprises a core onto which the aspirin and the tripotassium citrate monohydrate are coated.

In some embodiments, the water soluble analgesic composition further includes a surfactant. In certain of these embodiments, the surfactant comprises sodium lauryl sulfate. In some embodiments, the water soluble analgesic composition further includes a supplemental active ingredient selected from the group consisting of ascorbic acid, caffeine and combinations of these.

In accordance with another embodiment of the present invention, a water soluble analgesic composition includes aspirin and tripotassium citrate monohydrate, with a pH of the composition, when dissolved in water, being below about 6.0.

In some embodiments, the pH of the composition, when dissolved in water, falls within a range from about 5.2 to about 6.0. In certain of these embodiments, the pH of the composition, when dissolved in water, falls within a range from about 5.6 to about 6.0. In some embodiments, the aspirin comprises at least about 26% by weight of a combined weight of the aspirin and the tripotassium citrate monohydrate.

In some embodiments, the water soluble analgesic composition further includes a substrate. In certain of these embodiments, the substrate is selected from the group consisting of monosaccharides, disaccharides, polysaccharides, dipeptides and combinations of these. In certain of these embodiments, the substrate comprises sucrose. In some embodiments, the substrate comprises a core onto which the aspirin and the tripotassium citrate monohydrate are coated.

In some embodiments, the water soluble analgesic composition further includes a surfactant. In certain of these embodiments, the surfactant comprises sodium lauryl sulfate. In some embodiments, the water soluble analgesic composition further includes a supplemental active ingredient selected from the group consisting of ascorbic acid, caffeine and combinations of these.

In accordance with another embodiment of the present invention, a method of creating a water soluble analgesic composition includes the steps of: (i) providing aspirin, tripotassium citrate monohydrate, a surfactant, and a substrate, (ii) creating a first solution including the tripotassium citrate monohydrate, (iii) adding the aspirin to the first solution to create a second solution, (iv) adding the surfactant to the second solution, (v) filtering the second solution to remove residual amounts of the aspirin to create a filtered second solution, and (vi) spray drying the filtered second solution onto the substrate so as to form an agglomerated product comprising a plurality of granules. The aspirin comprises at least about 26% by weight of a combined weight of the aspirin and the tripotassium citrate monohydrate provided in step (i). A pH of the composition, when dissolved in water, is below about 6.0.

In some embodiments, the surfactant comprises sodium lauryl sulfate. In some embodiments, the substrate is selected from the group consisting of monosaccharides, disaccharides, polysaccharides, dipeptides and combinations of these. In certain of these embodiments, the substrate comprises sucrose. In some embodiments, the step of spray drying the filtered second solution onto a substrate employs a fluid-bed spray drying process. In some embodiments, the granules have a median diameter falling within a range from about 100μ to about 400μ. In certain of these embodiments, the granules have a median diameter of about 200μ.

In accordance with a further embodiment of the present invention, a rapidly dissolving composition comprising an aspirin salt is provided, wherein a portion of the composition containing 650 mg of aspirin is completely soluble in 100 ml of water in less than 60 seconds.

In some embodiments, the portion of the composition containing 650 mg of aspirin is completely soluble in 100 ml of water in less than 30 seconds. In certain of these embodiments, the portion of the composition containing 650 mg of aspirin is completely soluble in 100 ml of water in less than 15 seconds. In some embodiments, a pH of the composition, when dissolved in water, is below about 6.0. In certain of these embodiments, the pH of the composition, when dissolved in water, falls within a range from about 5.2 to about 6.0. In certain embodiments, the pH of the composition, when dissolved in water, falls within a range from about 5.6 to about 6.0.

The invention and its particular features and advantages will become more apparent from the following detailed description considered with reference to the accompanying drawings.

### BRIEF DESCRIPTION OF THE DRAWINGS

FIG. 1 graphically illustrates, based upon data collected from human patients, salicylate levels versus time for a water soluble aspirin composition in accordance with the present invention and for a well-known commercially available aspirin formulation.

FIG. 2 graphically illustrates the % product by weight as a function of median diameter of granules for a water soluble aspirin composition in accordance with the present invention.

FIGS. 3-6 show scanning electron micrographs of a water soluble aspirin composition in accordance with the present invention at different magnifications: FIG. 3 (magnification ruler: 290μ); FIG. 4 (magnification ruler: 140μ); FIG. 5 (magnification ruler: 20μ); and FIG. 6 (magnification ruler: 7.4μ).

FIG. 7 graphically illustrates the relationship between pH and % aspirin by weight of a combined weight of aspirin and tripotassium citrate monohydrate for a water soluble aspirin composition in accordance with the present invention.

### DETAILED DESCRIPTION OF AN EMBODIMENT OF THE INVENTION

The present invention satisfies the needs left unattained by the prior art, and is based, in part, upon the discovery that certain mixtures of aspirin with sodium lauryl sulfate (which serves as a surfactant), citrate salts, and disaccharides (such as sucrose), monosaccharides or other non-nutritive flavoring agents (which also serve as preservatives, antioxidants and demulcents) give aqueous solutions that are stable and have lower pH (specifically those that have pH in the range of 5.2-6.0) as compared to previously known formulations. This compares favorably to formulations prepared by the prior art which, when dissolved in water, are generally not palatable and give solutions with pH greater than 6.0. The novel formulations of the present invention, at lower pH, containing citrate, sodium lauryl sulfate, sucrose and aspirin are designed to be more readily absorbed since they are in a more undissociated form.

The major decomposition pathway of acetylsalicylic acid to salicylic acid and acetic acid is via hydrolysis. In the absence of water, decomposition of acetylsalicylic acid does not occur. It has been reported that hydrolysis of aspirin is reduced in the presence of citric acid. In addition, sodium lauryl sulfate has been reported to act both as a lubricant and a stabilizing agent. There is also an earlier report that sucrose hinders this decomposition pathway of acetylsalicylic acid, presumably by providing a protective layer that has low moisture content, which may protect the acetylsalicylic acid from hydrolysis. It is likely that the hydroxyl groups in sucrose are able to hydrogen bond with water and, thereby, provide a level of protection from hydrolysis to acetylsalicylic acid.

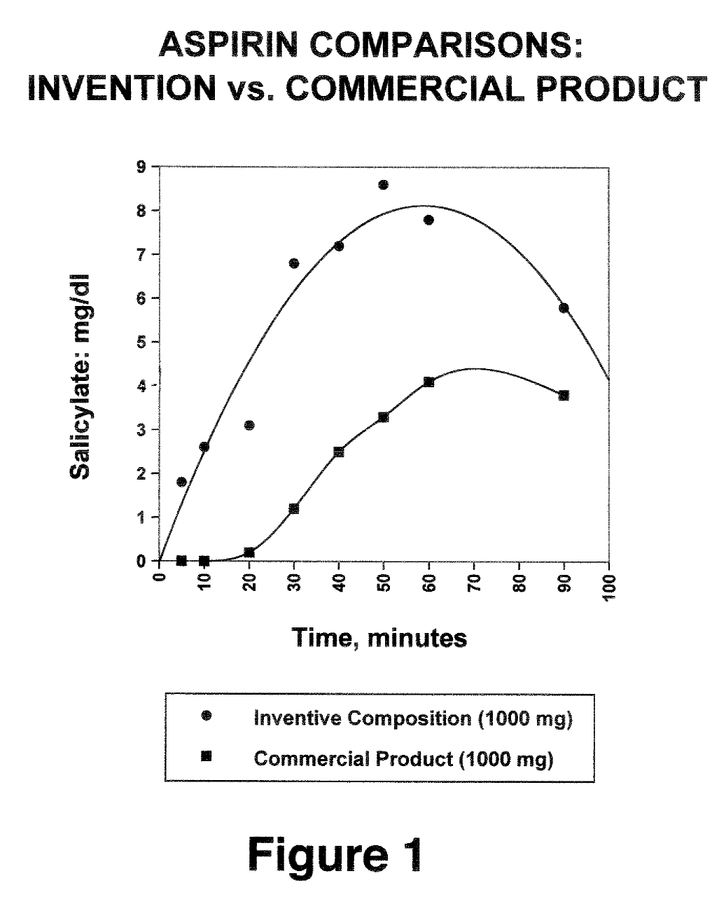

Administration of an aqueous solution of a water soluble aspirin composition in accordance with the instant invention gives higher levels of plasma salicylate concentrations compared to administration of aspirin in tablet or capsule form. FIG. 1 shows a graphic illustration, in which data collected from measurements of plasma salicylate levels in human patients is plotted for an aqueous solution of the inventive composition and for a known commercial product, specifically, Bayer® aspirin tablets. Both products were administered at the same 100 mg aspirin dose. Therapeutic levels of plasma salicylate were achieved within 5-10 minutes in accordance with the present invention, compared to 30-40 minutes for aspirin in tablet or capsule form. In addition, plasma salicylate levels were approximately twice as high for the inventive composition as compared to the commercial product. Thus, lower doses of the inventive composition can achieve comparable salicylate levels, and thereby minimize potential side effects of aspirin. The improved water solubility and palatability of the invention enables administration of larger doses of aspirin, as may be required for treating arthritis inflammation, while minimizing the potential gastric side-effects that are observed with commercially available aspirin in tablet or capsule form.

In accordance with one aspect of the invention, a method in accordance with which the ingredients are formulated is provided. To ensure that a homogeneous solution is obtained that rapidly dissolves in water and does not contain any particles of aspirin, the aspirin is first added to a solution of potassium citrate and sodium lauryl sulfate. Then, trace amounts of aspirin particles that have not been converted to its potassium salt are removed by filtration and the clear solution is then spray dried onto a core, such as crystalline sucrose, so as to form an agglomerated product. The use of a fluid-bed spray-drying process (a process using a combination of spray drying and agglomeration using air suspension technology) provides a coating of aspirin onto the sucrose core.

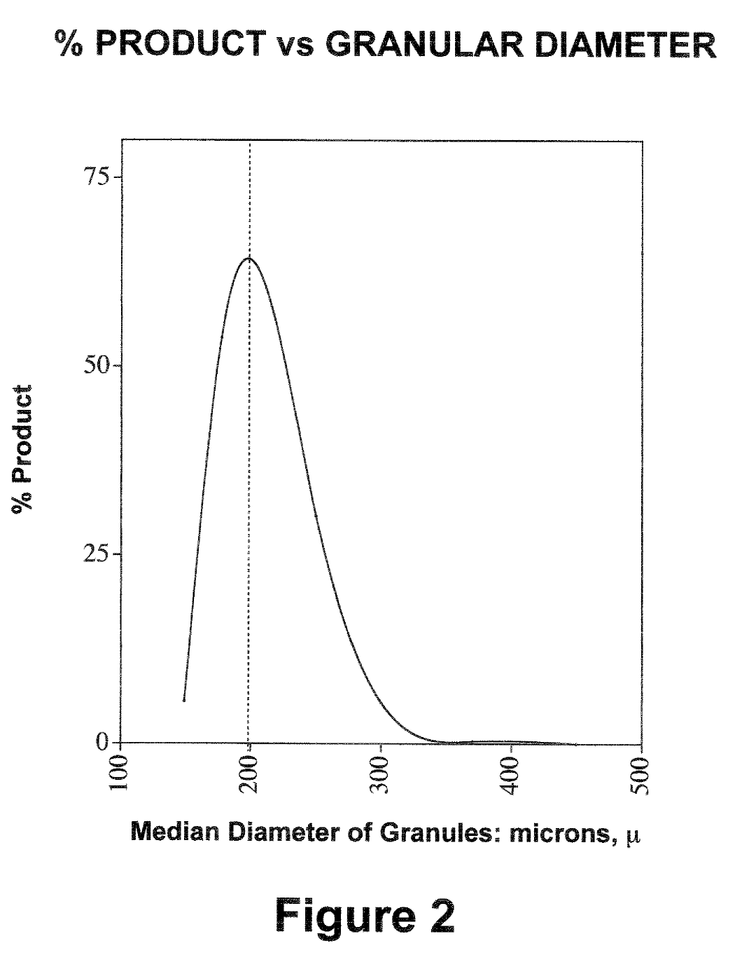

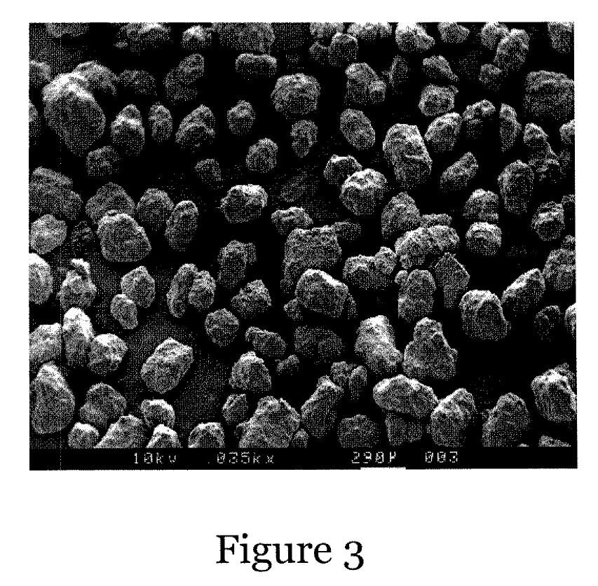

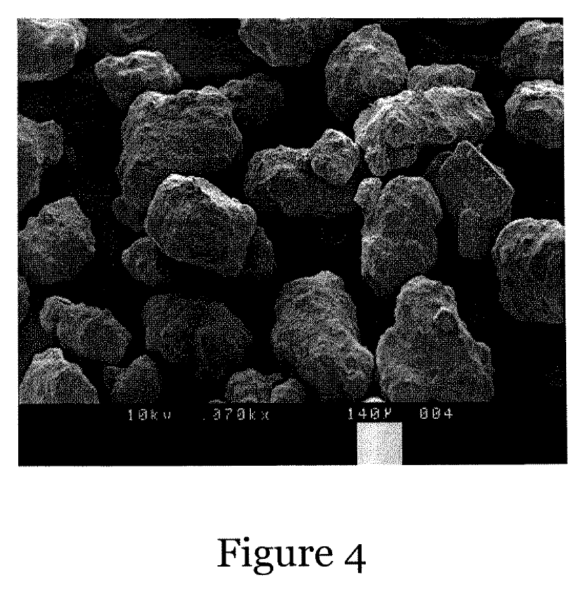

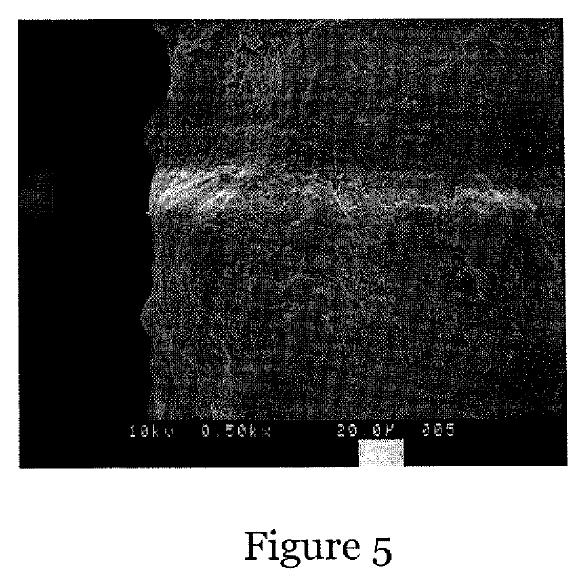

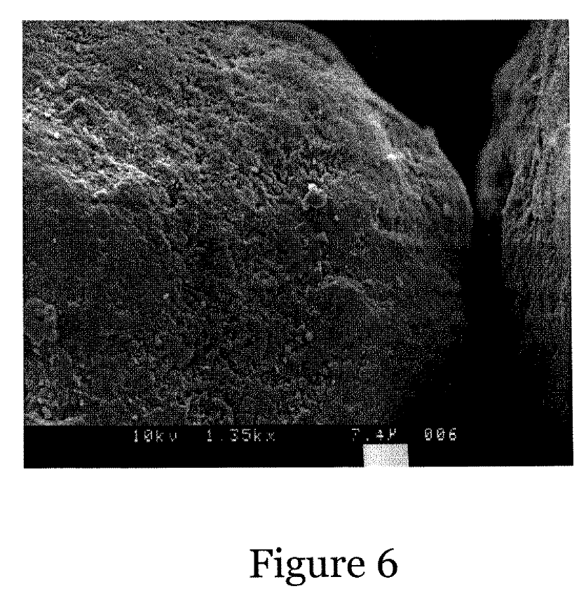

The resultant free-flowing solid formulation is freely soluble in water giving a clear, palatable aspirin solution (see Example 1 below). This granulation process provides product that contains granules of varying diameters ranging from about 100 to 400% with a median of about 200%, as illustrated in FIG. 2. These conclusions are confirmed by scanning electron microscopy, as shown in FIGS. 3-6, which illustrate the agglomerated product obtained from this process at varying magnification (FIG. 3 with the magnification ruler at 290μ, FIG. 4 with the magnification ruler at 140μ, FIG. 5 with the magnification ruler at 20.0μ, and FIG. 6 with the magnification ruler at 7.4μ).

Thus, the resultant free-flowing solid formulation contains a large number of granules, each of the granules consisting of a substrate (such as sucrose) and a coating agglomerated onto the substrate core. The coating includes a salt of aspirin, but substantially no particles of a nonsalt form of the aspirin. That is not to say that the coating includes no nonsalt form of the aspirin itself whatsoever, but rather that there are substantially no particles of the non-salt form of the aspirin contained in the coating, since substantially all of such particles are filtered during the process described above. Of course, the coating may include amounts of nonsalt form of the aspirin that had been previously dissolved in the solution before spray coating, since such dissolved amounts would not have been filtered as would particles thereof.

Formulations using sucrose or other non-nutritive sweeteners that are prepared directly and without incorporation of the fluid-bed spray-drying procedure also provide free-flowing products that are substantially soluble in water, but that may require a slightly longer time to dissolve completely (see Examples 2, 3 and 4 below).

The addition of certain supplemental active ingredients have been reported to enhance the beneficial effects of acetylsalicylic acid. For example, the combination of acetylsalicylic acid with ascorbic acid (Vitamin C) is rapidly transferred from the small intestines into the blood stream. This combination of aspirin and Vitamin C has been reported to be well suited for the treatment of headaches, pain and fever connected with colds. In addition, acetylsalicylic acid in combination with ascorbic acid has been reported to significantly reduces gastric lesion. The combination of Vitamin C with the novel formulation results in a product that is fully soluble in water (see Example 5 below).

The formulation is also completely compatible with the addition of caffeine, which has been reported to enhance the pain-relieving (analgesic) effects of acetylsalicylic acid, and has been proposed for use with other agents for the treatment of migraines. The combination of caffeine with the novel formulation results in a product that is fully soluble in water (see Example 6 below).

In addition to disaccharides, such as sucrose, other substrates, including monosaccharides, polysaccharides, dipeptides, etc. may be used in combination with acetylsalicylic acid in the novel formulation. There is an earlier report that the monosaccharide, D-glucose (dextrose), when used in combination with acetylsalicylic acid, has the added beneficial effect of reducing the gastrointestinal damage caused by analgesic pharmaceuticals. The formulation of aspirin with tripotassium citrate monohydrate, D-glucose and sodium lauryl sulfate was fully compatible and provided a homogeneous aqueous solution (see Example 7 below).

In addition, the monosaccharide, xylitol, has been reported to be useful in multilayered tablets containing aspirin, and may be used in the novel formulation (see Example 8 below). Cellulose, a polysaccharide that is insoluble in water, has been used in sustained-release tablet formulations of aspirin and may also be used in the novel formulation and pressed into pellets (see Example 9 below).

Following are several exemplary formulations of water soluble aspirin compositions in accordance with the present invention. It should be understood that the solubility tests described below were performed using deionized water and rapid magnetic stirring, that the tests were conducted at an ambient temperature of 20° C.±2° C., and that the portions of the inventive composition were added to the water all at once. It should also be understood that what is meant by the term “completely soluble” as used herein is that no particulates were visible to the naked eye in the solution resulting from the mixing of the inventive composition with water after the specified time period.

### EXAMPLE 1

Aspirin (625.0 g) was added portionwise to a solution of 1750.0 g of tripotassium citrate monohydrate in 10.0 L of water containing sodium lauryl sulfate (1.5 g). A trace amount of undissolved aspirin was removed by filtration. The resultant clear solution was slowly applied onto 2623.5 g of sucrose using a fluid-bed spray processor (inlet temperature: 45-47° C.; outlet temperature: 38-39° C.). The resulting agglomeration contained granulated product with a median particle size of about 200%. There was no detectable level of salicylic acid using the ferric chloride procedure, which can detect as little as 0.25% hydrolysis. A 5.2 g portion (containing 650 mg of aspirin) of the resultant free-flowing product in 100 ml of water with stirring and mixing was palatable and completely soluble within 15 seconds and gave a pH of 5.87.

### EXAMPLE 2

A mixture of 30.0 g of aspirin, 70.0 g of tripotassium citrate monohydrate, 100.0 g of sucrose and 60 mg of sodium lauryl sulfate was thoroughly shaken on a rocker assembly to ensure homogeneity. The resultant free-flowing product was stable for at least 3 weeks at 50° C. and at least 2 weeks at 75° C. and completely stable to ultraviolet light (254 nm) for at least 1 week. There was no detectable level of salicylic acid using the ferric chloride procedure, which can detect as little as 0.25% hydrolysis. Addition of 3.33 g of the mixture (containing 500 mg of aspirin) to 150 ml of purified water with stirring and mixing was palatable and substantially soluble within 15 seconds, completely soluble in 180 seconds, and gave a pH of 5.67.

### EXAMPLE 3

A mixture of 30.0 g of aspirin, 70.0 g of tripotassium citrate monohydrate, 20.0 g of aspartame and 36 mg of sodium lauryl sulfate was thoroughly shaken on a rocker assembly to ensure homogeneity. The resultant free-flowing product was stable for at least 3 weeks at 50° C. and at least 2 weeks at 75° C. There was no detectable level of salicylic acid using the ferric chloride procedure, which can detect as little as 0.25% hydrolysis. Addition of 2.00 g of the mixture (containing 500 mg of aspirin) to 150 ml of purified water with stirring and mixing was palatable and substantially soluble within 15 seconds, completely soluble in 240 seconds, and gave a pH of 5.93.

### EXAMPLE 4

A mixture of 30.0 g of aspirin, 70.0 g of tripotassium citrate monohydrate, 20.0 g of sucralose and 36 mg of sodium lauryl sulfate was thoroughly shaken on a rocker assembly to ensure homogeneity. The resultant free-flowing product was stable for at least 3 weeks at 50° C. There was no detectable level of salicylic acid using the ferric chloride procedure, which can detect as little as 0.25% hydrolysis. Addition of 2.00 g of the mixture (containing 500 mg of aspirin) to 150 ml of purified water with stirring and mixing was palatable and substantially soluble within 30 seconds, completely soluble in 210 seconds, and gave a pH of 5.74.

### EXAMPLE 5

A 4.75 g portion of the product from Example 1 (containing 561 mg of aspirin) was thoroughly mixed with 200 mg of Vitamin C. The resulting free-flowing product was dissolved in 100 ml of water with stirring and mixing. It was fully soluble within 30 seconds, gave a pH of 5.63, and was palatable.

### EXAMPLE 6

A 4.75 g portion of the product from Example 1 (containing 561 mg of aspirin) was thoroughly mixed with 50 mg of caffeine. The resulting free-flowing product was dissolved in 100 ml of water with stirring and mixing. It was fully soluble within 30 seconds, gave a pH of 5.86, and was palatable.

### EXAMPLE 7

A mixture of 11.8 g of aspirin, 33.1 g of tripotassium citrate monohydrate, 70.0 g of D-glucose (dextrose) and 30 mg of sodium lauryl sulfate was thoroughly shaken on a rocker assembly to ensure homogeneity, resulting in a free-flowing white product. There was no detectable level of salicylic acid using the ferric chloride procedure, which can detect as little as 0.25% hydrolysis. Addition of 3.16 g of the mixture to 38 ml of purified water with stirring was fully soluble within 30 seconds. This solution contained 1.93 g (5.0%) of D-glucose and 325 mg of aspirin, and had a pH of 5.84.

### EXAMPLE 8

A mixture of 4.8 g of aspirin, 13.4 g of tripotassium citrate monohydrate, 20.0 g of crystalline xylitol and 12 mg of sodium lauryl sulfate was thoroughly shaken on a rocker assembly to ensure homogeneity, resulting in a free-flowing white product. Addition of 2.60 g of the mixture (containing 325 mg of aspirin) to 100 ml of purified water with stirring and mixing was palatable and substantially soluble within 15 seconds, completely soluble in 60 seconds, and gave a pH of 5.99.

### EXAMPLE 9

A mixture of 4.8 g of aspirin, 13.4 g of tripotassium citrate monohydrate, 20.0 g of microcrystalline cellulose and 12 mg of sodium lauryl sulfate was thoroughly shaken on a rocker assembly to ensure homogeneity, resulting in a free-flowing white product. This product was insoluble in water, and was compressible into a pellet or wafer.

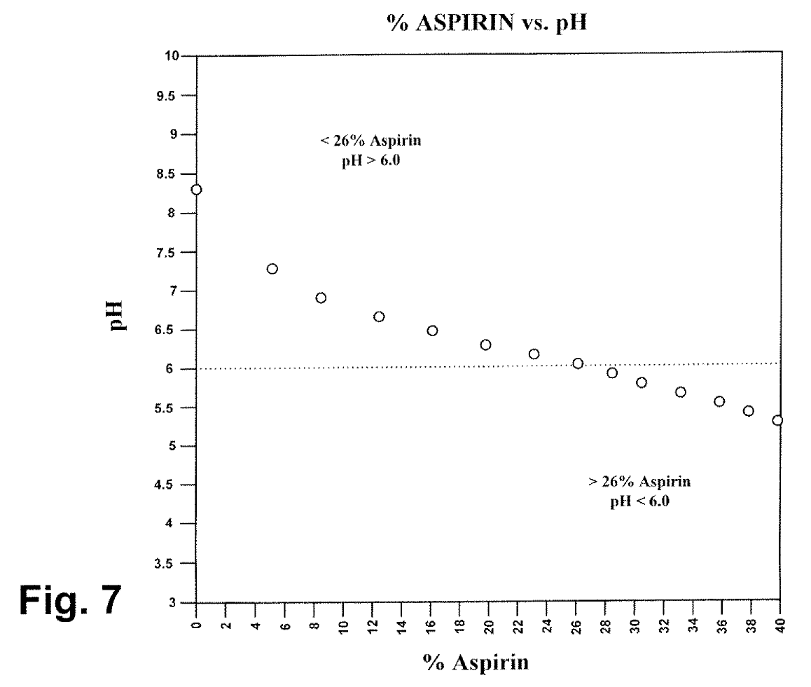

As can be seen from the above examples, the pH of each of the inventive compositions is below 6.0, which, as described above, provides a number of distinct advantages. It can be ensured to keep the pH in the desired range (i.e., <6.0) by varying the amount of aspirin in the composition as compared to the amount of tripotassium citrate monohydrate. More specifically, with an aspirin content of greater than about 26% by weight of a combined weight of aspirin and tripotassium citrate monohydrate (i.e., between 26% and 40% aspirin) the pH of the resulting solution is less than 6.0. For example, Example 1 above has approximately 26.3% aspirin content and has a pH of 5.87, while Example 2 above has approximately 30.0% aspirin content and has a pH of 5.67. On the other hand, at less than about 26% aspirin content (i.e., between 0 and 26% aspirin) the pH of the resulting solution is greater than 6.0. For example, it has been determined that Example 5 in U.S. Pat. No. 5,776,431 to Galat has about 20.0% aspirin content and has a pH of 6.12. The relationship between percent aspirin content and the resulting pH of the solution is graphically shown in FIG. 7.

The teachings, discoveries, procedures and methods described above, which specifically discuss acetylsalicylic acid (aspirin) as the active therapeutic in the formulations, are also applicable to other analgesics, and as such, the present invention is not limited to water soluble aspirin compositions, but rather encompasses water soluble analgesic compositions.

For example, the present invention encompasses formulations where water insoluble derivatives of salicylic acid are used as the active therapeutic. 5-Aminosalicylic acid (mesalamine), for example, is used to treat inflammatory bowel diseases, such as ulcerative colitis. Mesalamine is insoluble in water and is, therefore, usually used in extended release capsules or, alternatively, as a suppository. Typically, large daily doses of mesalamine (4 g/day) are required for treatment of inflammatory bowel diseases. It has been reported that the solubility-pH profile of mesalamine is increased at pH <2.0 and pH >5.5. Formulations of mesalamine in accordance with the teachings of the present invention result in a pH of 6.86, which results in a homogeneous aqueous solution that is fast acting and enters the blood stream rapidly (see Example 10 below). The formulation is palatable and may include a variety of substrates, including sucrose.

Other water insoluble analgesics, including acetaminophen (see Example 11 below), ibuprofen (see Example 12 below) and naproxen (see Example 13 below), were prepared using the novel formulation procedure.

Following are several exemplary formulations of water soluble analgesic compositions in accordance with the present invention that employ analgesics other than acetylsalicylic acid (aspirin) as the active therapeutic.

### EXAMPLE 10

A mixture of 800 mg of mesalamine, 10.0 g of tripotassium citrate monohydrate, 14.92 g of sucrose and 8 mg of sodium lauryl sulfate was thoroughly shaken on a rocker assembly to ensure homogeneity, resulting in a free-flowing off-white product. Addition of 6.39 g of the mixture (containing 325 mg of mesalamine) to 100 ml of purified water with stirring was mostly soluble within 15 seconds and completely soluble within 25 seconds. This solution had a pH of 6.86 and was palatable.

### EXAMPLE 11

A mixture of 1.20 g of acetaminophen, 3.35 g of tripotassium citrate monohydrate, 5.0 g of sucrose and 3 mg of sodium lauryl sulfate was thoroughly shaken on a rocker assembly to ensure homogeneity, resulting in a free-flowing white product. Addition of 2.7 g of the mixture (containing 325 mg of acetaminophen) to 100 ml of purified water with stirring was mostly soluble within 15 seconds and fully soluble in 45 seconds. This solution had a pH of 7.80 and was palatable.

### EXAMPLE 12

A mixture of 125 mg of ibuprofen, 2.50 g of tripotassium citrate monohydrate, 3.73 g of sucrose and 2 mg of sodium lauryl sulfate was thoroughly shaken on a rocker assembly to ensure homogeneity, resulting in a free-flowing white product. Addition of the mixture (containing 125 mg of ibuprofen) to 75 ml of purified water with stirring was substantially soluble within 15 seconds and completely soluble in 240 seconds. This solution had a pH of 7.23 and was palatable.

### EXAMPLE 13

A mixture of 125 mg of naproxen, 2.50 g of tripotassium citrate monohydrate, 3.73 g of sucrose and 2 mg of sodium lauryl sulfate was thoroughly shaken on a rocker assembly to ensure homogeneity, resulting in a free-flowing white product. Addition of the mixture (containing 125 mg of ibuprofen) to 75 ml of purified water with stirring was substantially soluble within 15 seconds and completely soluble in 60 seconds. This solution had a pH of 7.40 and was palatable.

Since water soluble analgesic compositions in accordance with the present invention employ known analgesics, the compositions are anticipated to be used to prevent and treat substantially all known conditions, diseases, types of patients, etc. currently treated using the known formulations of these analgesics. However, given the many benefits of water soluble analgesic compositions in accordance with the present invention discussed above, it is anticipated that such compositions will have even a wider range of applications.

The present invention, therefore, provides a water soluble analgesic composition which has enhanced stability and bioactivity as compared to previously known water soluble analgesic compositions, which is sodium free, which is rapidly water soluble, which is fast acting and enters the bloodstream rapidly, and which may be used in the relatively large dosages that are required for anti-inflammatory treatment, and/or that may be used for extended periods of time, without causing gastrointestinal upset and/or damage.

Although the invention has been described with reference to a particular arrangement of parts, features and the like, these are not intended to exhaust all possible arrangements or features, and indeed many other modifications and variations will be ascertainable to those of skill in the art.

What is claimed is:

1. A water soluble analgesic composition comprising a plurality of granules, each of the granules comprising:
a substrate core; and
a coating disposed on the substrate core forming an agglomerated product, said coating comprising a salt of an analgesic, but substantially no particles of a non-salt form of the analgesic.

2. The water soluble analgesic composition of claim 1 wherein said substrate core is selected from the group consisting of monosaccharides, disaccharides, polysaccharides, dipeptides and combinations of these.

3. The water soluble analgesic composition of claim 2 wherein said substrate core comprises sucrose.

4. The water soluble analgesic composition of claim 1 wherein the granules have a median diameter falling within a range from about 100μ to about 400μ.

5. The water soluble analgesic composition of claim 4 wherein the granules have a median diameter of about 200μ.

6. The water soluble analgesic composition of claim 1 wherein the analgesic is selected from the group consisting of aspirin, 5-aminosalicylic acid, ibuprofen, naproxen, acetaminophen and combinations of these.

7. The water soluble analgesic composition of claim 6 wherein the analgesic comprises aspirin.

8. The water soluble analgesic composition of claim 1 wherein the salt of the analgesic comprises a potassium salt of the analgesic.

9. A method of creating a water soluble analgesic composition comprising the steps of:
providing a first solution comprising a base;
adding an analgesic to the first solution to create a second solution comprising a salt of the analgesic;
filtering the second solution to remove residual particles of the analgesic to create a filtered second solution; and
spray drying the filtered second solution onto a substrate so as to form an agglomerated product comprising a plurality of granules.

10. The method of claim 9 wherein the analgesic is selected from the group consisting of aspirin, 5-aminosalicylic acid, ibuprofen, naproxen, acetaminophen and combinations of these.

11. The method of claim 10 wherein the analgesic comprises aspirin.

12. The method of claim 9 wherein the base comprises tripotassium citrate monohydrate.

13. The method of claim 9 wherein the first solution further comprises a surfactant.

14. The method of claim 13 wherein the surfactant comprises sodium lauryl sulfate.

15. The method of claim 9 wherein the substrate is selected from the group consisting of monosaccharides, disaccharides, polysaccharides, dipeptides and combinations of these.

16. The method of claim 15 wherein the substrate comprises sucrose.

17. The method of claim 9 wherein said step of spray drying the filtered second solution onto a substrate employs a fluid-bed spray drying process.

18. The method of claim 9 wherein the granules have a median diameter falling within a range from about 100μ to about 400μ.

19. The method of claim 18 wherein the granules have a median diameter of about 200μ.

20. A water soluble analgesic composition comprising:
aspirin;
tripotassium citrate monohydrate; and
wherein said aspirin comprises at least about 26% by weight of a combined weight of said aspirin and said tripotassium citrate monohydrate.

21. The water soluble analgesic composition of claim 20 wherein said aspirin comprises from about 26% to about 40% by weight of a combined weight of said aspirin and said tripotassium citrate monohydrate.

22. The water soluble analgesic composition of claim 20 wherein a pH of said composition, when dissolved in water, is below about 6.0.

23. The water soluble analgesic composition of claim 20 further comprising a substrate.

24. The water soluble analgesic composition of claim 23 wherein said substrate is selected from the group consisting of monosaccharides, disaccharides, polysaccharides, dipeptides and combinations of these.

25. The water soluble analgesic composition of claim 24 wherein said substrate comprises sucrose.

26. The water soluble analgesic composition of claim 23 wherein said substrate comprises a core onto which said aspirin and said tripotassium citrate monohydrate are coated.

27. The water soluble analgesic composition of claim 20 further comprising a surfactant.

28. The water soluble analgesic composition of claim 27 wherein said surfactant comprises sodium lauryl sulfate.

29. The water soluble analgesic composition of claim 20 further comprising a supplemental active ingredient selected from the group consisting of ascorbic acid, caffeine and combinations of these.

30. A water soluble analgesic composition comprising:
aspirin;
tripotassium citrate monohydrate; and
wherein a pH of said composition, when dissolved in water, is below about 6.0.

31. The water soluble analgesic composition of claim 30 wherein the pH of said composition, when dissolved in water, falls within a range from about 5.2 to about 6.0.

32. The water soluble analgesic composition of claim 31 wherein the pH of said composition, when dissolved in water, falls within a range from about 5.6 to about 6.0.

33. The water soluble analgesic composition of claim 30 wherein said aspirin comprises at least about 26% by weight of a combined weight of said aspirin and said tripotassium citrate monohydrate.

34. The water soluble analgesic composition of claim 30 further comprising a substrate.

35. The water soluble analgesic composition of claim 34 wherein said substrate is selected from the group consisting of monosaccharides, disaccharides, polysaccharides, dipeptides and combinations of these.

36. The water soluble analgesic composition of claim 35 wherein said substrate comprises sucrose.

37. The water soluble analgesic composition of claim 34 wherein said substrate comprises a core onto which said aspirin and said tripotassium citrate monohydrate are coated.

38. The water soluble analgesic composition of claim 30 further comprising a surfactant.

39. The water soluble analgesic composition of claim 38 wherein said surfactant comprises sodium lauryl sulfate.

40. The water soluble analgesic composition of claim 30 further comprising a supplemental active ingredient selected from the group consisting of ascorbic acid, caffeine and combinations of these.

41. A method of creating a water soluble analgesic composition comprising the steps of:
providing aspirin, tripotassium citrate monohydrate, a surfactant, and a substrate, wherein said aspirin comprises at least about 26% by weight of a combined weight of said aspirin and said tripotassium citrate monohydrate;
creating a first solution comprising the tripotassium citrate monohydrate;
adding the aspirin to the first solution to create a second solution;
adding the surfactant to the second solution;
filtering the second solution to remove residual amounts of the aspirin to create a filtered second solution;
spray drying the filtered second solution onto the substrate so as to form an agglomerated product comprising a plurality of granules; and
wherein a pH of said composition, when dissolved in water, is below about 6.0.

42. The method of claim 41 wherein the surfactant comprises sodium lauryl sulfate.

43. The method of claim 41 wherein the substrate is selected from the group consisting of monosaccharides, disaccharides, polysaccharides, dipeptides and combinations of these.

44. The method of claim 43 wherein the substrate comprises sucrose.

45. The method of claim 41 wherein said step of spray drying the filtered second solution onto a substrate employs a fluid-bed spray drying process.

46. The method of claim 41 wherein the granules have a median diameter falling within a range from about 100μ to about 400μ.

47. The method of claim 46 wherein the granules have a median diameter of about 200μ.

48. A rapidly dissolving composition comprising an aspirin salt, wherein a portion of said composition containing 650 mg of aspirin is completely soluble in 100 ml of water in less than 60 seconds.

49. The rapidly dissolving composition of claim 48 wherein the portion of said composition containing 650 mg of aspirin is completely soluble in 100 ml of water in less than 30 seconds.

50. The rapidly dissolving composition of claim 49 wherein the portion of said composition containing 650 mg of aspirin is completely soluble in 100 ml of water in less than 15 seconds.

51. The rapidly dissolving composition of claim 48 wherein a pH of said composition, when dissolved in water, is below about 6.0.

52. The rapidly dissolving composition of claim 51 wherein the pH of said composition, when dissolved in water, falls within a range from about 5.2 to about 6.0.

53. The rapidly dissolving composition of claim 52 wherein the pH of said composition, when dissolved in water, falls within a range from about 5.6 to about 6.0.
